(19) United States
(12) Patent Application Publication
(10) Pub. No.: US 2014/0187860 A1
(43) Pub. Date: Jul. 3, 2014

(54) GUIDABLE ENDOSCOPE

(71) Applicants: METAL INDUSTRIES RESEARCH & DEVELOPMENT CENTRE, KAOHSIUNG (TW); TAIPEI VETERANS GENERAL HOSPITAL, TAIPEI CITY (TW)

(72) Inventors: Tung-Chieh YANG, PINGTUNG COUNTY (TW); Yi-Hsiu HUANG, TAIPEI CITY (TW); Tsung-Chih YU, TAINAN CITY (TW)

(73) Assignees: METAL INDUSTRIES RESEARCH & DEVELOPMENT CENTRE, KAOHSIUNG (TW); TAIPEI VETERANS GENERAL HOSPITAL, TAIPEI CITY (TW)

(21) Appl. No.: 14/141,437

(22) Filed: Dec. 27, 2013

(30) Foreign Application Priority Data
Dec. 28, 2012 (TW) 101151067

(51) Int. Cl. A61B 1/00; A61B 1/307

(52) U.S. Cl. CPC A61B 1/00071; A61B 1/307
USPC 600/114

(57) ABSTRACT

A guidable endoscope is described, which includes a rigid guiding part, a bending controllable part, a rigid endoscope body and a handle part. The rigid guiding part has a first end and a second end opposite to each other. The bending controllable part has a first end and a second end opposite to each other. The first end of the bending controllable part is directly connected to the second end of the rigid guiding part. The rigid endoscope body has a first end and a second end opposite to each other. The first end of the rigid endoscope body is directly connected to the second end of the bending controllable part, and the bending controllable part can be bent relative to the rigid guiding part or the rigid endoscope body. The handle part is connected to the second end of the rigid endoscope body.

### RELATED APPLICATIONS

This application claims priority to Taiwan Application Serial Number 101151067, filed Dec. 28, 2012, which is herein incorporated by reference.

### FIELD OF THE INVENTION

The present invention relates to an endoscope, and more particularly to a guidable endoscope.

### BACKGROUND OF THE INVENTION

Endoscopes are one kind of common medical instrument device, which are medical instrument entering a human body through various tracts of the human body for the medical staff to observe an internal condition of the human body. There are various kinds of endoscopes, and some of which also have treatment functions, such as cystoscopes, gastroscopes and colonoscopes.

Taking a urethroscope as an example, the urethroscope is the most common endoscope while examining and treating a urinary system. The urethroscope is mainly used to observe that whether a calculus is formed in a urinary tract of a human body or not, and to remove the calculus from the urinary tract of the human body by taking out or breaking the calculus. The ureteral calculus is an agglomerated lump formed of crystalline substances secreted by a kidney. According to where the calculus is formed, the calculus may be distinguished into a calculus of kidney, a calculus of ureter, a vesical calculus and a calculus of urethra.

The operation of the urethroscope now encounters some problems. For the ureteral orifice or a physiological narrow region, it is not easy for the urethroscope set to get into, and a guide wire is needed for guiding. In addition, when the ureter cyst is swollen, a polypus grows in the ureter or the ureter is convulsive, a visual field of the urethroscope is obstructed, so that the guide wire is additionally used to guide the urethroscope into the sufferer. Furthermore, the viewable visual fields of some urethroscopes cannot be adjusted, thereby resulting in blind entrance of the urethroscopes to injure a mucosa tissue of a tract of a human body. Moreover, a structure of an upper ureter system, such as a kidney, is complicated, so that it is hard for the urethroscopes to enter to obverse, and to make a further diagnosis and give treatment.

In addition, a tube of the urethroscopes is not formed of a memory material, so that the tube of the urethroscopes is bent and damaged after the diagnosis and the treatment. Accordingly, the medical treatment cost is greatly increased. Some urethroscopes are designed to have a thin front end and a thick rear end. However, such a urethroscope is stuck in a ureteral orifice while making a further diagnosis and giving treatment, and the urethroscope pulls apart an internal tissue of a human body while retreating.

### SUMMARY OF THE INVENTION

Therefore, one aspect of the present invention is to provide a guidable endoscope, in which a front end is set with a rigid guiding part, and a bending controllable part follows the rigid guiding part. Therefore, the front end of the guidable endoscope has a guiding function without a guide wire. In addition, with the bending controllable part, the rigid guiding part on the front end has swing ability, so that a visual field during diagnosis and treatment is increased to enhance efficiency of a surgical operation, and injury to a patient tissue and damage to the endoscope are decreased to greatly mitigate complications of the patient after the surgical operation and to reduce a medical treatment cost.

Another aspect of the present invention is to provide a guidable endoscope, in which a memory material may be used as a material of a tube of a rigid guiding part, so that it can effectively prevent the rigid guiding part from being bent and damaged after a surgical operation due to compression of a human body internal tissue.

Still another aspect of the present invention is to provide a guidable endoscope, in which a bending controllable part can support a rigid guiding part and can make a front end of the endoscope be partially bent to get into a zigzag ureter. Therefore, it can prevent the endoscope from getting into a human body blindly to injure an internal mucosa tissue of a patient body.

Further another aspect of the present invention is to provide a guidable endoscope, in which an endoscope body comprising a rigid guiding part, a bending controllable part and a rigid endoscope body has a uniform diameter, so that it can prevent the endoscope from being stuck in a ureteral orifice while making a diagnosis and giving treatment, and pulling apart an internal tissue of a human body while retreating the endoscope.

According to the aforementioned objectives, the present invention provides a guidable endoscope. The guidable endoscope includes a rigid guiding part, a bending controllable part, a rigid endoscope body and a handle part. The rigid guiding part has a first end and a second end opposite to each other. The bending controllable part has a first end and a second end opposite to each other. The first end of the bending controllable part is directly connected to the second end of the rigid guiding part. The rigid endoscope body has a first end and a second end opposite to each other. The first end of the rigid endoscope body is directly connected to the second end of the bending controllable part, and the bending controllable part can be bent in relation to the rigid guiding part or the rigid endoscope body. The handle part is connected to the second end of the rigid endoscope body.

According to a preferred embodiment of the present invention, a length of the rigid guiding pan is from 1 cm to 10 cm.

According to another preferred embodiment of the present invention, a diameter of the rigid guiding part, a diameter of the bending controllable part and a diameter of the rigid endoscope body are equal to each other.

According to still another preferred embodiment of the present invention, each of the rigid guiding part and the rigid endoscope body includes a tube, and a material of the tube includes an elastic memory material.

According to further another preferred embodiment of the present invention, the material of the tube is a medical metal, and the medical metal includes a NiTi alloy.

According to yet another preferred embodiment of the present invention, the bending controllable part includes a first layer, a second layer and a third layer stacked in sequence from inside to outside. The first layer includes as spiral metal sheet, the second layer includes as metal braided mesh, and the third layer includes a flexible crackless material film.

According to still further another preferred embodiment of the present invention, a bending dimension of the bending controllable part is one-dimensional, two-dimensional or three-dimensional.

According to still yet another preferred embodiment of the present invention, a bending angle of the bending controllable part is from −15 degrees to +15 degrees.

BRIEF DESCRIPTION OF THE DRAWINGS

The foregoing aspects and many of the attendant advantages of this invention are more readily appreciated as the same become better understood by reference to the following detailed description, when taken in conjunction with the accompanying drawings, wherein:

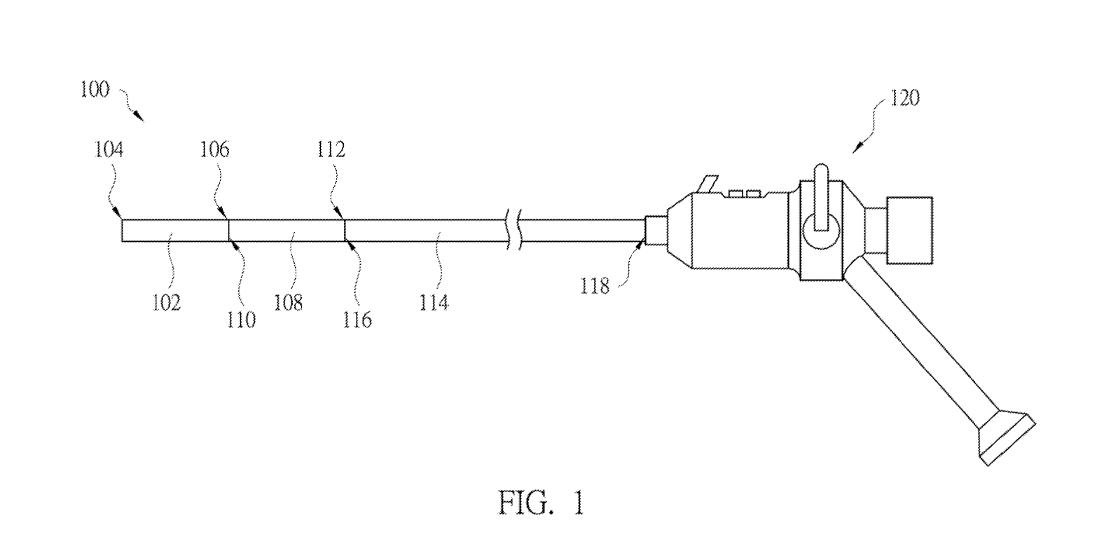

FIG. 1 illustrates a side view of a guidable endoscope in accordance with an embodiment of the present invention;

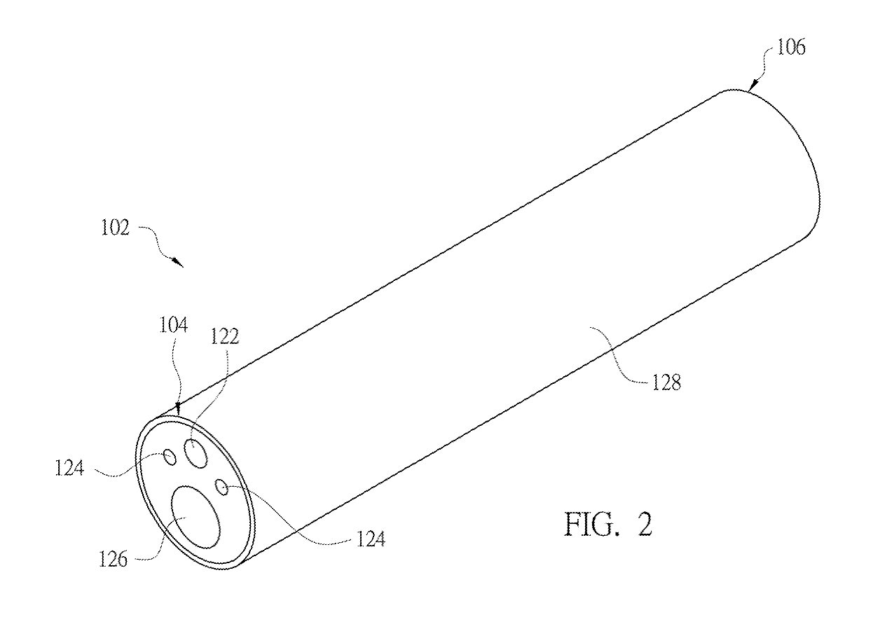

FIG. 2 is a three-dimensional diagram showing a rigid guiding part in accordance with an embodiment of the present invention;

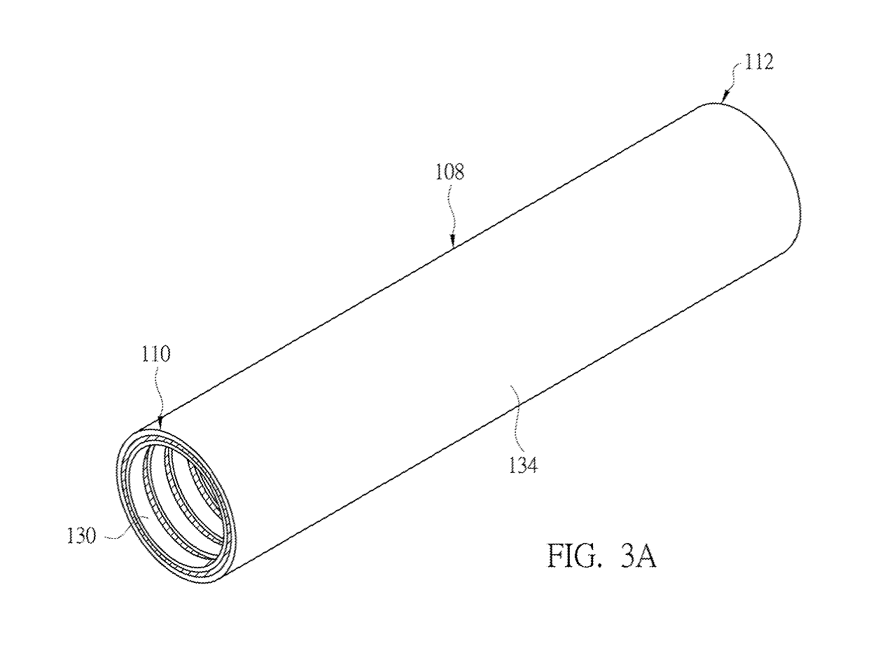

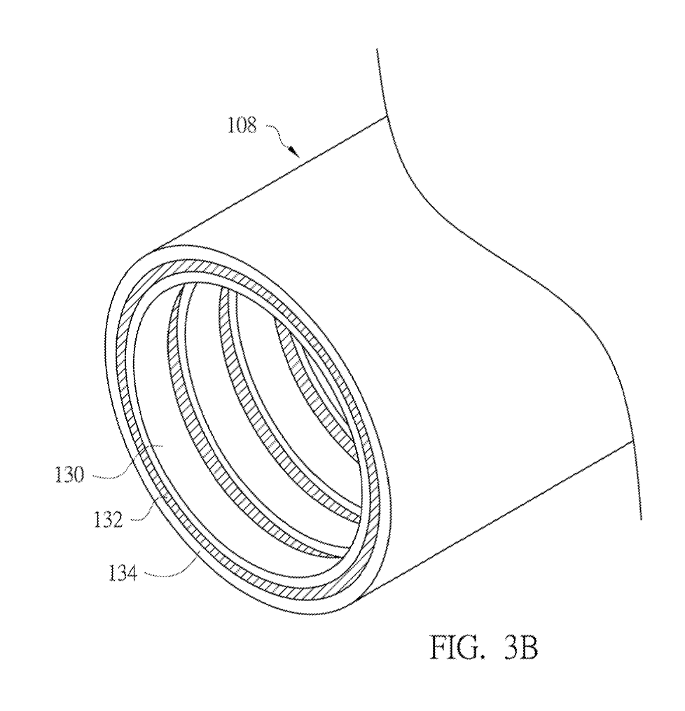

FIG. 3A is a three-dimensional diagram showing a bending controllable part in accordance with an embodiment of the present invention;

FIG. 3B illustrates a partially enlarged view of a structure of a bending controllable part in accordance with an embodiment of the present invention;

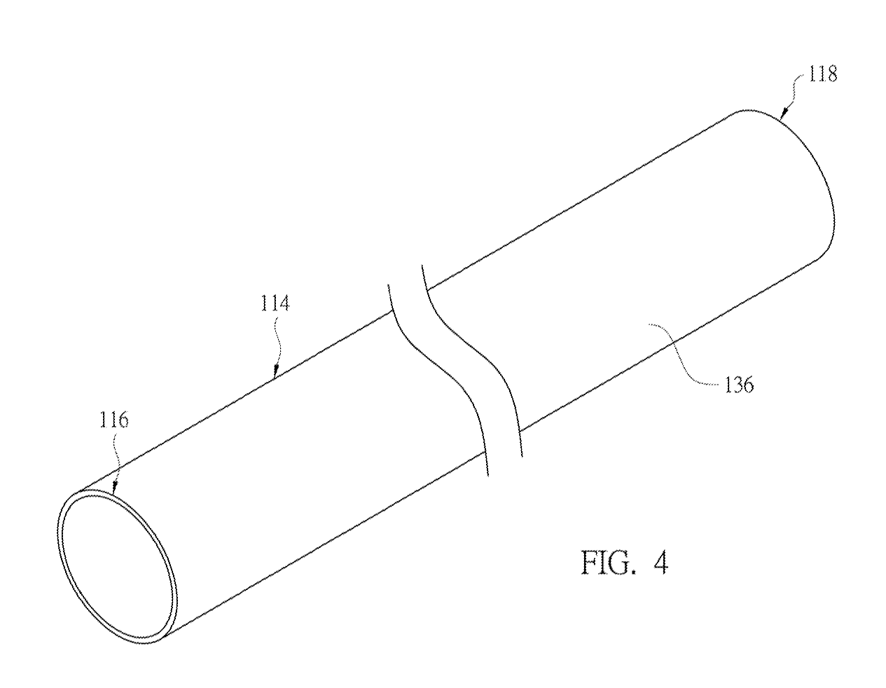

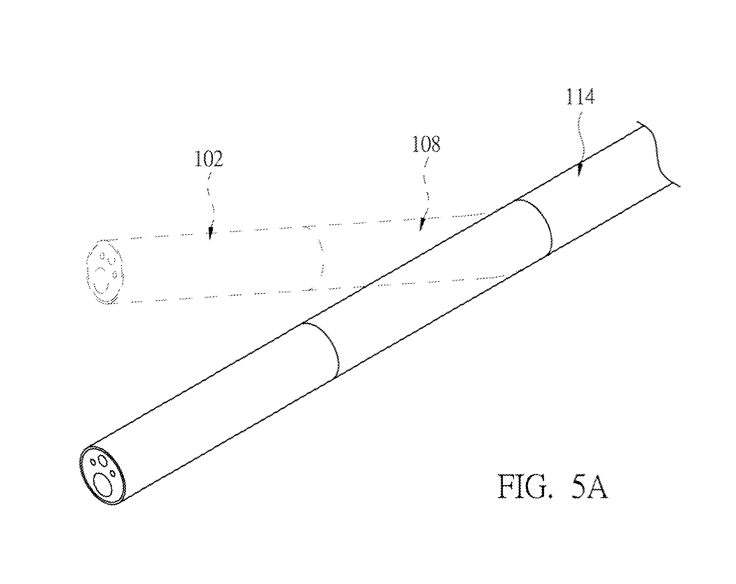

FIG. 4 is a three-dimensional diagram showing a rigid endoscope body in accordance with an embodiment of the present invention;

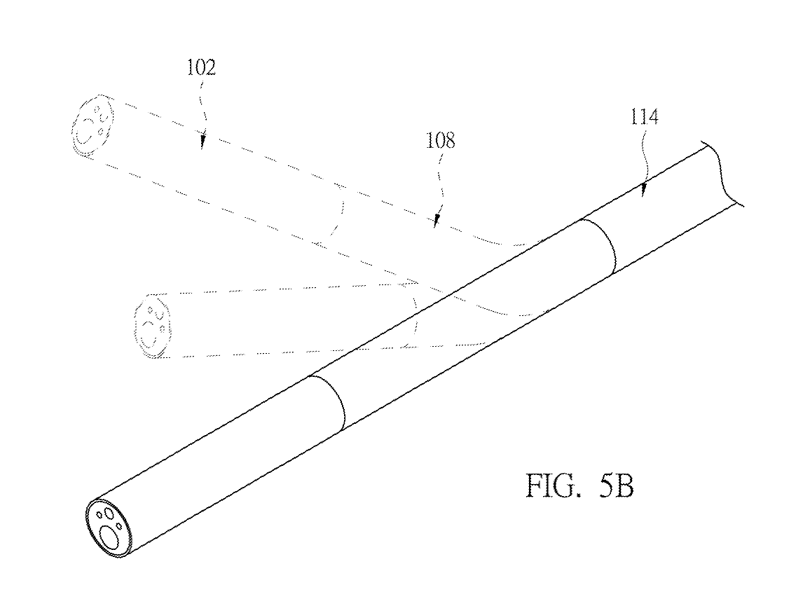

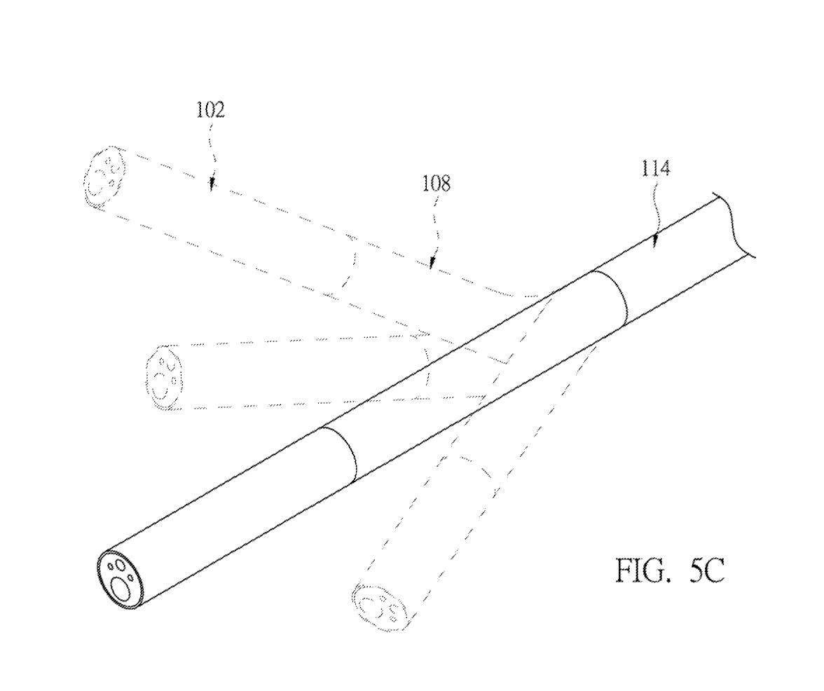

FIG. 5A is a schematic diagram showing a bending controllable part having one-dimensional bending dimension in accordance with an embodiment of the present invention;

FIG. 5B is a schematic diagram showing a bending controllable part having two-dimensional bending dimension in accordance with another embodiment of the present invention;

FIG. 5C is a schematic diagram showing a bending controllable part having three-dimensional bending dimension in accordance with still another embodiment of the present invention; and

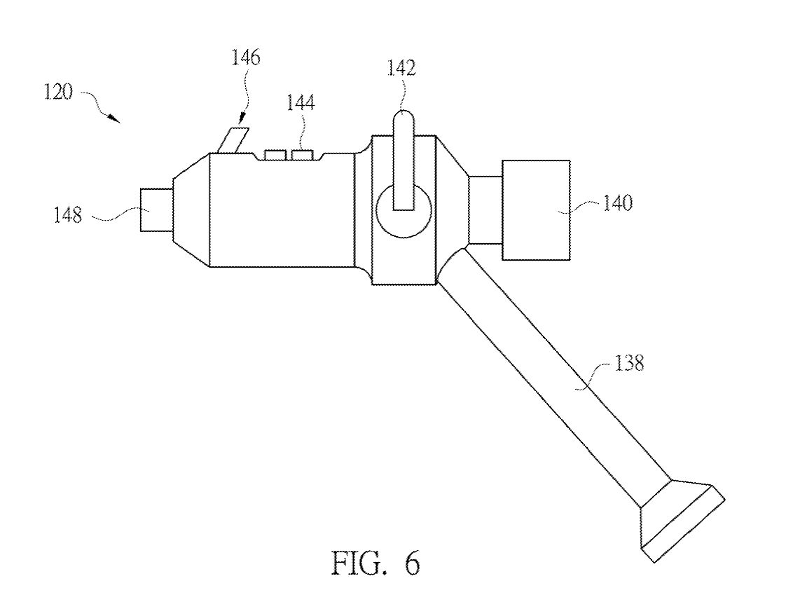

FIG. 6 is a schematic diagram showing a handle part in accordance with an embodiment of the present invention.

### DETAILED DESCRIPTION OF THE PREFERRED EMBODIMENT

Refer to FIG. 1. FIG. 1 illustrates a side view of a guidable endoscope in accordance with an embodiment of the present invention. In the present embodiment, a guidable endoscope 100 is suitable to observe urinary systems of human bodies for further diagnoses and treatments of the urinary systems. The guidable endoscope 100 may mainly include a rigid guiding part 102, a bending controllable part 108, a rigid endoscope body 114 and a handle part 120.

Refer to FIG. 2 simultaneously. FIG. 2 is a three-dimensional diagram showing a rigid guiding part in accordance with an embodiment of the present invention. In the guidable endoscope 100, the rigid guiding part 102 has a first end 104 and a second end 106. The first end 104 and the second end 106 are respectively located on two sides of the rigid guiding part 102. The first end 104 is the end adjacent to sufferers. In one exemplary embodiment, a length of the rigid guiding part 102 may be, for example, from 1 cm to 10 cm to keep the rigidness of the rigid guiding part 102 for easy guiding. In one preferred embodiment, the length of the rigid guiding part 102 may be, for example, from 2 cm to 4 cm.

In one exemplary embodiment, the rigid guiding part 102 may include a tube 128. A material of the tube 128 may include an elastic material with memory, and with rigidness preferably. With the use of the elastic material with memory and rigidness, it can prevent the endoscope body of the guidable endoscope 100 from being bent after use to reduce the medical treatment cost. In one example, the material of the tube 128 may use a medical metal, and the medical metal may include, for example, a NiTi alloy.

In one exemplary embodiment, the rigid guiding part 102 may further include an objective lens 122, at least one light source 124 and at least one work channel 126. The objective lens 122, the at least one light source 124 and the at least one work channel 126 are all disposed within the tube 128 of the rigid guiding part 102. The objective lens 122 may be composed of a photosensitive device and a prism set. The photosensitive device may be, for example, a charge coupled device (CCD) or a complementary metal-oxide semiconductor (CMOS) photosensitive device.

The light source 124 may be a light-emitting diode (LED), and may be directly disposed within the tube 128 via a connection wire In another exemplary embodiment, the light-emitting diode may be disposed on a back end of the guidable endoscope 100, and the light source 124 may further include a light-guiding optical fiber for transmitting light emitted from the light-emitting diode on the back end to the rigid guiding part 102 on the front end. The work channel 126 is a passageway for instruments used during the diagnosis and treatment process, such as clamps, electrotomes, baskets, electrodes or lasers, and fluids, such as water and gas, to get in and out.

Simultaneously refer to FIG. 3A and FIG. 3B. FIG. 3A and FIG. 3B are respectively a three-dimensional diagram showing a bending controllable part in accordance with an embodiment of the present invention, and a partially enlarged view of a structure of the bending controllable part. The bending, controllable part 108 has a first end 110 and a second end 112, such as shown in FIG. 3A. The first end 110 and the second end 112 are respectively located on two opposite ends of the bending controllable part 108. As shown in FIG. 1, the first end HO of the bending controllable part 108 is directly connected to the second end 106 of the rigid guiding part 102. In one embodiment, the first end 110 of the bending controllable part 108 and the second end 106 of the rigid guiding part 102 may be combined by inserting, hitching, covering, pasting, attaching, adhering, spirally fastening, nailing, tenoning, embedding and welding, for example.

As shown in FIG. 3B, the bending controllable part 108 may be a tri-layered structure, for example. The tri-layered structure includes a first layer 130, a second layer 132 and a third layer 134 stacked in sequence from inside to outside. In one example, the first layer 130 may be a spiral metal sheet, in which spiral gaps in the spiral metal sheet are provided for facilitating the structural curving of the spiral metal sheet. The second layer 132 may include a metal braided mesh. The structural strength of the spiral metal sheet can be strengthened by putting the metal braided mesh to cover an outer surface of the spiral metal sheet. In addition, with the application of the metal braided mesh, the covering area of the third layer 134 can be increased to enhance the adhesive force of the third layer 134 to the second layer 132 and the first layer 130.

The third layer 134 may be mainly used to prevent, liquid and gas permeating into the bending controllable part 108. The third layer 134 may include a flexible crackless material film, in which the flexible crackless material film preferably has a high temperature and high pressure resistant property. In one exemplary embodiment, a material of the flexible crackless material film may be metal or polymer by one exemplary example, polyvinyl chloride (PVC) may be used as a material of the third layer 134 due to good softness, corrosion resistance, high temperature resistance, wear resistance and pulling resistance of PVC.

In another exemplary embodiment., the bending controllable part 108 may he a circular-ring-shaped flexible tube, for example. The circular-ring-shaped flexible tube is a tubular shell including ripples, each of which is in a closed circular ring shape, in which any two adjacent wave structures of the circular-ring-shaped flexible tube may be serially connected by the circular-ring-shaped ripple. The circular-ring-shaped flexible tube may be formed by processing a crackless tube or a welded tube. The circular-ring-shaped flexible tube may be a flexible structure composed of metal or plastic. In one example, the circular-ring-shaped flexible tube may be a circular-ring-shaped stainless steel flexible tube.

Refer to FIG. 4. FIG. 4 is a three-dimensional diagram showing a rigid endoscope body in accordance with an embodiment of the present invention. The rigid endoscope body 114 similarly has a first end 116 and a second end 118. The first end 116 and the second end 118 are respectively located on two opposite ends of the rigid endoscope body 114. As shown in FIG. 1, the first end 116 of the rigid endoscope body 114 is directly connected to the second end 112 of the bending controllable part 108. In one exemplary embodiment, the first end 116 of the rigid endoscope body 114 and the second end 112 of the bending controllable part 108 may be combined similarly by inserting, hitching, covering, pasting, attaching, adhering, spirally fastening, nailing, tenoning, embedding and welding, for example. The rigid guiding part 102, the bending controllable part 108 and the rigid endoscope body 114 form an inset part of the guidable endoscope 100, i.e. all the rigid guiding part 102, the bending controllable part 108 and the rigid endoscope body 114 are the parts that can enter a body of a patient.

The rigid endoscope body 114 includes a tube 136. A material of the tube 136 may include an elastic memory material, and is preferably with rigidness. By adopting the elastic memory material with rigidness, it can prevent the guidable endoscope 100 from being bent after using, thereby can achieve the purpose of improving the medical treatment convenience. In one example, the material of the tube 136 may adopt a medical metal, and the medical metal may include a NiTi alloy, for example.

In one embodiment, a diameter of the rand guiding part 102 a diameter of the bending controllable part 108 and a diameter of the rigid endoscope body 114 may be equal to each other. Therefore, the diameter of the inset part of the guidable endoscope 100 is uniform to prevent the guidable endoscope 100 from being stuck in a ureteral orifice while making a further diagnosis and giving treatment, and pulling apart an internal tissue of a human body while retreating the guidable endoscope 100.

In the guidable endoscope 100, the rigidness of the rigid guiding part 102 and the rigid endoscope body 114 is much larger than that of the bending controllable part 108 and the bending controllable part 108 is located between the rigid guiding part 102 and the rigid endoscope body 114. Accordingly, the bending controllable part 108 can be bent in relation to the rigid guiding part 102 or the rigid endoscope body 114, so that the rigid guiding part 102 on the front end has swing ability. Therefore, with the bending controllable part 108, a visual field during diagnosis and treatment is increased to enhance efficiency of the diagnosis and the treatment, and injury to a patient tissue and damage to the endoscope are decreased.

In the present embodiment, the bending controllable part 108 may be controlled by a mechanically-driven method or a power-driven method. A bending degree of the bending controllable part 108 may be adjusted according to application requirements. In one exemplary embodiment, the guidable endoscope 100 is a urethroscope, and the bending controllable part 108 of the guidable endoscope 100 does not need to have a bending degree with a very large angle, which is different from a laparoscope or an arthroscope that needs a very large bending angle. A bending angle of the bending controllable part 108 may be, for example, from −30 degrees to +30 degrees, and preferably from −15 degrees to +15 degrees.

Furthermore, refer to FIG. 5A to FIG. 5C. FIG. 5A to FIG. 5C are respectively schematic diagrams showing bending controllable parts having one-dimensional bending dimension, two-dimensional bending dimension and three-dimensional bending dimension, in accordance with embodiments of the present invention. The bending dimension of the bending controllable part 108 can be designed according to application requirements. The bending controllable part 108 may have one-dimensional bending dimension such as shown in FIG. 5A, may have two-dimensional bending dimension such as shown in FIG. 5B, or may have three-dimensional bending dimension such as shown in FIG. 5C. The bending controllable part 108 having multi-dimensional bending dimensions is more flexible. With the flexibility and the support of the bending controllable part 108, the existing of the rigid guiding part 102 can make the guidable endoscope 100 have a guiding function and a diagnosis and treatment function while performing an upper ureter surgery.

Refer to FIG. 6. FIG. 6 is a schematic diagram showing a handle part in accordance with an embodiment of the present invention. In one exemplary embodiment, the handle part 120 may include a hand grip 138, an image/light source terminal 140, a control bending part 142, a control button 144, a work channel inlet 146 and a support part 148. The support part 148 is disposed on a front end of the handle part 120 to connect with the inset part of the guidable endoscope 100 composed of the rigid guiding part 102, the bending controllable part 108 and the rigid endoscope body 114. That is the second end 118 of the rigid endoscope body 114 is connected to the support part 148 of the handle part 120, such as shown in FIG. 1 and FIG. 6. In one exemplary embodiment, the second end 118 of the rigid endoscope body 114 and the support part 148 may be combined b inserting, bitching, pasting, attaching, adhering, spirally fastening, nailing, tenoning and welding, for example.

The image/light source terminal 140 ma be disposed on a back end of the handle part 120 to facilitate the electrical connection with external accessory instruments to output images and supply electric power. The light source 124 and the objective lens 122 may be equipped with connection wires being connected to the image/light source terminal 140 of the handle part 120 of the guidable endoscope 100, and the electric power and the images are respectively supplied and received by the external accessory instruments. The control bending part 142 may be disposed on a top end of the handle part 120 to facilitate the operation of the bending of the bending controllable part 108. The hand grip 138 may be disposed on a lower end of the handle part 120 to facilitate an operator to hold the guidable endoscope 100. The control button 144 may be disposed on the top end of the handle part 120 to control the operation of the photosensitive devices and the light source. The work channel inlet 146 may be similarly disposed on the top end of the handle part 120 for the instruments and the fluids used during the diagnosis and treatment process to get into the work channel 126.

According to the aforementioned embodiments of the present invention, one advantage of the present invention is that a front end is set with a rigid guiding part, and a bending controllable part follows the rigid guiding part, so that the front end of the guidable endoscope has a guiding function without a guide wire, in addition, with the bending controllable part, the rigid guiding part on the front end has swing, ability, so that a visual field during diagnosis and treatment is increased to enhance efficiency of a surgical operation, and injury to a patient tissue and damage to the endoscope are decreased to greatly mitigate complications of the patient after the surgical operation and to reduce a medical treatment cost.

According to the aforementioned embodiments of the present invention, another advantage of the present invention is that a memory material may be used as a material of a tube of a rigid guiding part of a guidable endoscope, so that it can effectively prevent the rigid guiding pan from being bent and damaged after a surgical operation due to compression of a human body internal tissue.

According to the aforementioned embodiments of the present invention, still another advantage of the present invention is that a bending controllable part of a guidable endoscope can support a rigid guiding part and can make a front end of the endoscope be partially bent to get into a zigzag ureter. Therefore, it can prevent the endoscope from getting into a human body blindly to injure an internal mucosa tissue of a patient body.

According to the aforementioned embodiments of the present invention, further another advantage of the present invention is that an endoscope body of to guidable endoscope comprising a rigid guiding part, a bending controllable part and a rigid endoscope body has a uniform diameter, so that it can prevent the endoscope from being stuck in a ureteral orifice while making a diagnosis and giving treatment, and pulling apart an internal tissue of a human body while retreating the endoscope.

As is understood by a person skilled in the art, the foregoing preferred embodiments of the present invention are illustrative of the present invention rather than limiting of the present invention. It is intended to cover various modifications and similar arrangements included within the spirit and scope of the appended claims, the scope of which should be accorded the broadest interpretation so as to encompass all such modifications and similar structure.

What is claimed is:

1. A guidable endoscope, including:
a rigid guiding part having a first end and a second end opposite to each other;
a bending controllable part having a first end and a second end opposite to each other, wherein the first end of the bending controllable part is directly connected to the second end of the rigid guiding part;
a rigid endoscope body having a first end and a second end opposite to each other, wherein the first end of the rigid endoscope body is directly connected to the second end of the bending controllable pail, and the bending controllable part can be bent in relation to the rigid guiding part or the rigid endoscope body; and
a handle part connected to the second end of the rigid endoscope body.

2. The guidable endoscope according to claim 1, wherein a length of the rigid guiding part is from 1 cm to 10 cm.

3. The guidable endoscope according to claim 1, wherein a diameter of the rigid guiding part, a diameter of the bending controllable part and a diameter of the rigid endoscope body are equal.

4. The guidable endoscope according to claim 1, wherein each of the rigid guiding part and the rigid endoscope body includes a tube, and a material of the tube includes an elastic memory material.

5. The guidable endoscope according to claim 4, wherein the material of the tube is a medical metal, and the medical metal includes NiTi alloy.

6. The guidable endoscope according to claim 1, wherein the bending controllable part includes a first layer, a second layer and a third layer stacked in sequence from inside to outside.

7. The guidable endoscope according to claim 6, wherein the first layer includes a spiral metal sheet, the second layer includes a metal braided mesh, and the third layer includes a flexible crackless material film.

8. The guidable endoscope according to claim 1, wherein a bending dimension of the bending controllable part is one-dimensional, two-dimensional or three-dimensional.

9. The guidable endoscope according to claim 1, wherein a bending angle of the bending controllable part is from −15 degrees to +15 degrees.
